(19) United States
(12) Patent Application Publication
(10) Pub. No.: US 2020/0306071 A1
(43) Pub. Date: Oct. 1, 2020

(54) Inflatable Brace

(71) Applicant: Aero Med Design Tech LLC, Salt Lake City, UT (US)

(72) Inventors: Carl Yeip, Salt Lake City, UT (US); Matthew Fuller, Salt Lake City, UT (US)

(21) Appl. No.: 16/624,553

(22) PCT Filed: Jun. 19, 2018
(86) PCT No.: PCT/US2018/038378

§ 371 Date: Dec. 19, 2019

Related U.S. Application Data
(60) Provisional application No. 62/522,596, filed on Jun. 20, 2017.
(60) Provisional application No. 62/640,462, filed on Mar. 8, 2018.

(51) Int. Cl. A61F 5/058; A61F 5/02; A61F 5/055

(52) U.S. Cl. CPC A61F 5/05816; A61F 5/055; A61F 5/02

(57) ABSTRACT

An inflatable brace. The inflatable brace including a flexible variably inflatable membrane elongated in a first direction and a second direction, the flexible variably inflatable membrane being fastenable around an object and inflatable to provide particular structural support to the object and an inflatable channel disposed between a first layer of the variably inflatable membrane and a second layer of the variably inflatable membrane, the inflatable channel comprising a predetermined arrangement of segregated inflatable sub-channels that are segregated by a plurality of dividers, the arrangement of segregated inflatable sub-channels, when inflated, providing the particular structural support to the object.

### CROSS-REFERENCE TO RELATED APPLICATIONS

The present application claims the benefit under 35 U.S.C. § 119(e) of U.S. Provisional Patent Application Ser. No. 62/640,462, titled “Inflatable Appendage Brace,” filed on Mar. 08, 2018, the entire contents of which are incorporated herein by reference. The present application also claims the benefit under 35 U.S.C. § 119(e) of U.S. Provisional Patent Application Ser. No. 62/522,596, titled “Inflatable Cervical Collar,” filed on Jun. 20, 2017, the entire contents of which are incorporated herein by reference.

### BACKGROUND

This application relates to inflatable braces, and in a more particular non-limiting example embodiment, to inflatable appendage braces, such as an inflatable cervical collar.

Appendage braces are used to provide structural support to an appendage of a patient. Appendage braces provide this support using rigid materials, such as molded plastics, to provide the support to the appendage of a patient. However, appendage braces as a result of the nature of having to provide support to an appendage are rigid and cannot be easily stored and moved. Furthermore, appendage braces are difficult to use because they require positioning of the appendage brace around the appendage, often when the appendage has suffered an injury. The jostling and movements caused by using the appendage brace may further aggravate any injuries to the appendage.

One type of appendage brace that suffers from these limitations is a cervical collar. A cervical collar, also known as a neck brace, is a medical device used to support a person's neck. A cervical collar may be used by emergency service personnel on patients who have had traumatic head or neck injuries to prevent further injuries and stabilize existing injuries. The cervical collar can also be used to treat chronic medical conditions.

When a patient has a potential traumatic head or neck injury, they may suffer from a cervical spine fracture. This creates a high-risk situation placing them at greater risk for spinal cord injury, which could be further exacerbated by moving the patient and can ultimately lead to paralysis or death. Examples of this type of injury could include a person suspected of having whiplash because of a car accident, a skier suffering a hard fall on a ski slope, or a hiker falling from a cliff. Such patients may have a rigid protective cervical collar placed by a medical professional until radiographic imaging can be taken to determine if the patients have suffered a cervical spine fracture or other injury. The cervical collar serves to stabilize the top seven vertebrae, C1 through C7 (other immobilizing devices such as a backboard can be used to stabilize the remainder of the spinal column).

Existing cervical collars suffer from a number of drawbacks. In particular, cervical collars are designed to be rigid and made of rigid materials, such as hard metal and/or plastic. These rigid cervical collars are bulky and cumbersome because of the shape. Therefore, few emergency medical technicians (“EMT”s) will carry them in an emergency situation. As such, cervical collars are stored in vehicles (such as fire trucks or ambulances) rather than on an EMT or other medical responder's person. Furthermore, these rigid cervical collars are cumbersome to carry when an EMT is required to be mobile (e.g., ski patrol, search and rescue, National Park Service, etc.). This is due, in large part, to the fact that the collar is bulky and rigid. Indeed, if there is a presumed need for a cervical collar, and access to the patient is difficult or in a challenging environment, made more difficult by the fact that speed is crucial in an emergency, the EMT will often clip the rigid cervical collar to the outside of all other gear. This may lead to further issues as the rigid cervical collar is more likely to be damaged when it bounces around on the outside of all the other gear. Rigid cervical collars also can cause pressure sores where the edges of the rigid cervical collar meet a patient's body as a result of the rigid material that presses against the patient's body to keep it immobilized. Rigid cervical collars can also be difficult to place on a patient and require movements of the patient (when any movement can potentially exacerbate a spinal injury) to get the rigid cervical collar around the patient's neck and size the rigid cervical collar to immobilize the patient.

### SUMMARY

The techniques introduced herein overcome the deficiencies and limitations of the prior art, at least in part, with an inflatable braces, such as an inflatable appendage brace.

One innovative aspect includes an inflatable brace that comprises a flexible variably inflatable membrane elongated in a first direction and a second direction. The flexible variably inflatable membrane may be fastenable around an object and inflatable to provide particular structural support to the object. The flexible variably inflatable membrane may include an inflatable channel disposed between a first layer of the variably inflatable membrane and a second layer of the variably inflatable membrane. The inflatable channel includes a predetermined arrangement of segregated inflatable sub-channels that are segregated by a plurality of dividers. The arrangement of segregated inflatable sub-channels, when inflated, provide the particular structural support to the object.

Various implementations may optionally include one or more of the following features. The inflatable brace may include an inflation mechanism coupled to the flexible variably inflatable membrane. The inflation mechanism may cause the inflatable channel to be inflated. The inflation mechanism may be located on a front side of the variably inflatable membrane and configured to provide symmetrical inflation of the inflatable channel. The plurality of dividers may include at least one u-shaped divider that prevents folding of the flexible variable inflatable membrane when the inflatable channel is inflated. The predetermined arrangement of segregated inflatable sub-channels may include at least one of a first horizontal inflatable sub-channel and a second horizontal inflatable sub-channel. The inflatable brace may include the first horizontal inflatable sub-channel being disposed along a top portion of the flexible inflatable membrane in the first direction and adjacent to a top of at least a portion of the plurality of dividers. The inflatable brace may include the second horizontal inflatable sub-channel being disposed along a bottom portion of the flexible inflatable membrane in the first direction and adjacent to a bottom of at least a portion of the plurality of dividers. The flexible variably inflatable membrane may further includes an opening such that a target area of the object can be accessed through the opening when the flexible variably inflatable membrane is fastened around the object. The flexible variably inflatable membrane may further include a shelf formed out of a portion of a top edge of the flexible variably inflatable membrane. The shelf may be configured to provide specific structural support to retain a protrusion of the object. The inflatable channel may be configured to provide particular structural support to a target area of the object. The flexible variably inflatable membrane is fastenable around the object using a fastener. The fastener may be coupled to a first end portion of the variably inflatable membrane and configured to couple with a second end portion of the variably inflatable membrane and retain the variably inflatable membrane in a tubular shape around the object. The fastener may comprise one of a Velcro patch and webbing configured to be threaded through a slit of the variable inflatable membrane. The second material may have a second elasticity that allows for the second material to stretch more in the first direction of the variably inflatable membrane than in the second direction. The first layer of the variably inflatable membrane includes a first material having a first elasticity and the second layer of the variably inflatable membrane includes a second material having a second elasticity greater than the first elasticity. The second elasticity provides for an increased stretching of the second material when the inflatable channel disposed between the first layer and the second layer is inflated. The flexible variably inflatable membrane may be configured to lie substantially flat and/or be rolled, stuffed, or balled up when the inflatable channel is uninflated. The flexible variably inflatable membrane further includes instructions displayed on an exterior portion of the flexible variably inflatable membrane which include information on how to use the inflatable brace. The flexible variably inflatable membrane is capable of being rolled up on itself for storage when the inflatable channel is uninflated. The first layer of the variably inflatable membrane includes a first material having a first elasticity and the second layer of the variably inflatable membrane includes a second material having a second elasticity greater than the first elasticity. The second elasticity provides for an increased stretching of the second material when the inflatable channel disposed between the first layer and the second layer is inflated.

Another innovative aspect of the present disclosure includes a method of using an inflatable brace. The method includes unrolling a flexible variably inflatable membrane in a deflated state. The flexible variably inflatable membrane may be elongated in a first direction. The method further includes: sliding a first end of the flexible variably inflatable membrane in the deflated state under an object; fastening the flexible variably inflatable membrane in the deflated state around the object by fastening the first end of the flexible variably inflatable membrane to a second end of the flexible variably inflatable membrane; centering an access area formed out of a front portion of the flexible variably inflatable membrane around an access point of the object; and inflating an inflatable channel of the flexible variably inflatable membrane into an inflated state to provide particular structural support to the object. The particular structural support being formed by a predetermined arrangement of segregated inflatable sub-channels that are segregated by a plurality of dividers.

Another innovative aspect of the present disclosure includes an inflatable brace that comprises a flexible variably inflatable membrane and an inflatable channel. The flexible variably inflatable membrane may be elongated in a first direction and a second direction. The flexible variably inflatable membrane may be fastenable around a neck of a user and inflatable to provide particular structural support to the neck of the user. The inflatable channel may be disposed between a first layer of the variably inflatable membrane and a second layer of the variably inflatable membrane. The inflatable channel includes a predetermined arrangement of segregated inflatable sub-channels that are segregated by a plurality of dividers. The plurality of dividers may include a plurality of vertical dividers and one or more variably-shaped dividers (e.g,. u-shaped). The arrangement of segregated inflatable sub-channels, when inflated, may provide the particular structural support to the neck of the user.

Various implementations may optionally include one or more of the following features. The flexible variably inflatable membrane further includes an opening that provides access to the neck of the patient when the flexible variably inflatable membrane is inflated. The flexible variably inflatable membrane may be fastenable around the neck of the user using a fastener. The fastener may be coupled to a first end portion of the variably inflatable membrane and configured to couple with a second end portion of the variably inflatable membrane and retain the variably inflatable membrane in a tubular shape around the object.

Another innovative aspect of the present disclosure includes a method of manufacturing an inflatable brace, the method including: identifying a first shape of a first layer of a flexible variably inflatable membrane and identifying a second shape of a second layer of a flexible variably inflatable membrane using a predetermined pattern, cutting, with a cutting tool, the first layer and the second layer to the shape identified by the first shape and the second shape, joining the first layer with the second layer in the predetermined pattern, preparing the flexible variably inflatable membrane for a valve attachment, and installing the valve attachment.

The features and advantages described herein are not all-inclusive and many additional features and advantages will be apparent to one of ordinary skill in the art in view of the figures and description. Moreover, it should be noted that the language used in the specification has been principally selected for readability and instructional purposes and not to limit the scope of the techniques described.

### BRIEF DESCRIPTION OF THE DRAWINGS

The techniques introduced herein are illustrated by way of example, and not by way of limitation in the figures of the accompanying drawings in which like reference numerals are used to refer to similar elements.

FIG. 1 shows an example embodiment of an inflatable brace positioned on a user.

FIG. 2 shows an example embodiment of an inflatable brace

FIG. 3 shows an example embodiment of an inflatable brace positioned around an object.

FIG. 4 shows a top view of an example embodiment of an inflatable brace fastened around itself

FIGS. 5A and 5B show example embodiments of an inflatable brace from a front and a back view.

FIGS. 6A and 6B show example embodiments of an inflatable brace.

FIG. 7 shows an example embodiment of an inflatable brace positioned on a user.

FIG. 8 shows a flowchart of an example method of applying and inflating an inflatable brace.

FIG. 9 shows a flowchart of an example method of manufacturing an inflatable brace.

The Figures depict various embodiments for purposes of illustration only. One skilled in the art will readily recognize from the following discussion that alternative embodiments of the examples depicted herein may be employed without departing from the principles described herein.

### DETAILED DESCRIPTION

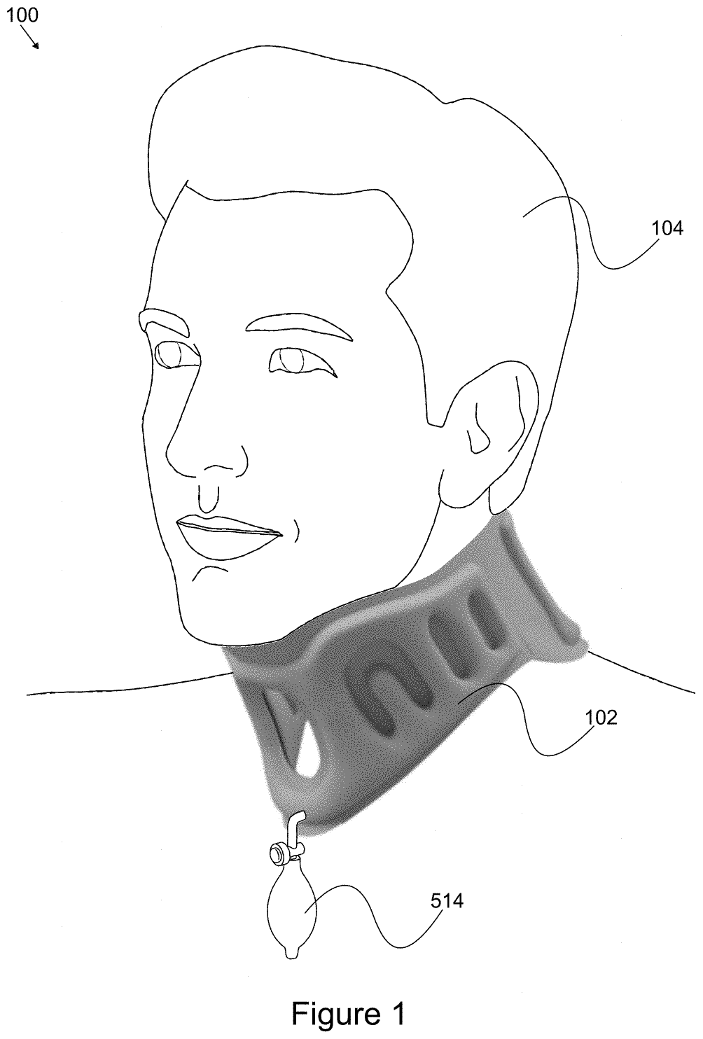

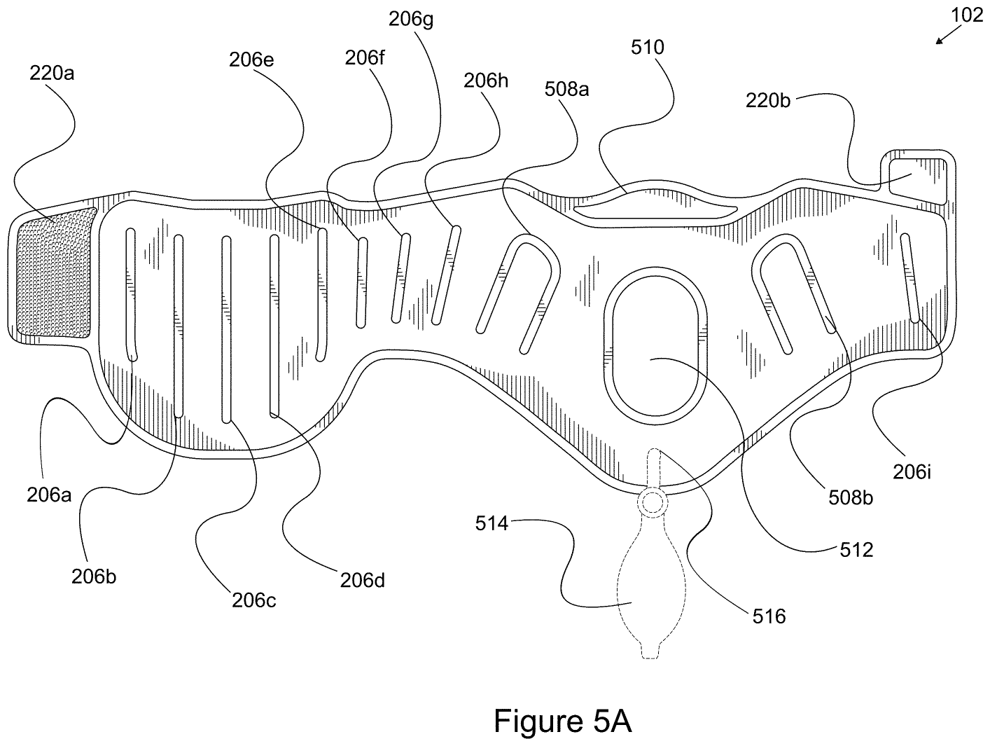

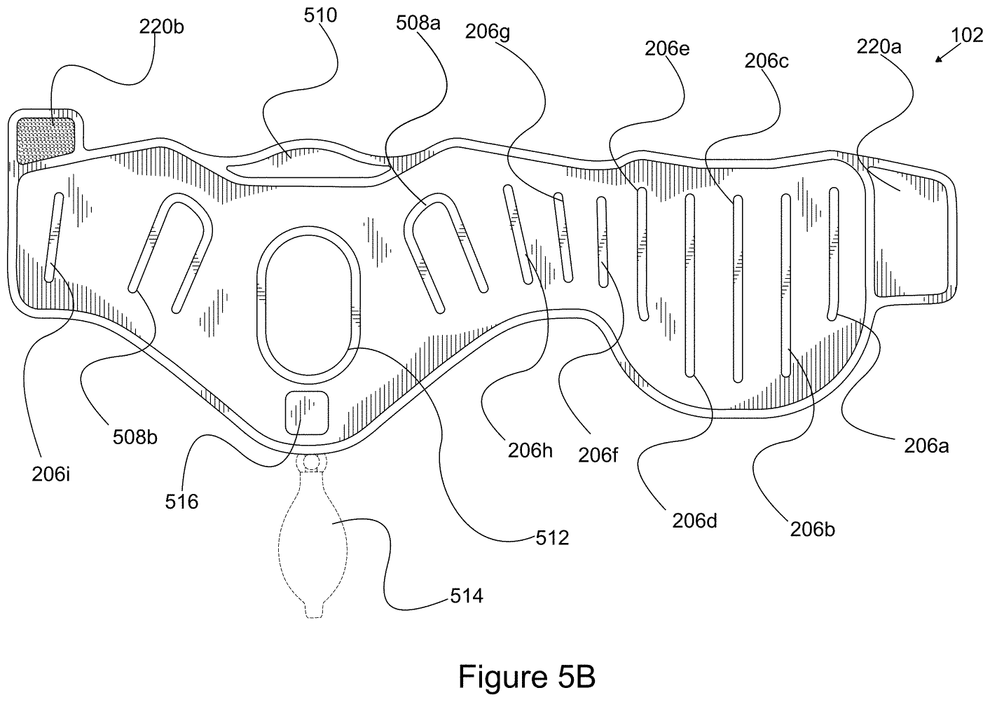

FIG. 1 shows an example embodiment 100 of an inflatable brace 102 immobilizing an appendage of a user 104. The inflatable brace 102 may be applied to immobilize of movement of a specific appendage of the user 104. For example, if the user 104 has potentially suffered an injury to the upper spine, the inflatable brace 102 may be used to immobilize the person's neck and spine while the user 104 is transported to a treatment facility. In another example, if the user 104 has potentially suffered an injury to another appendage, such as an arm, knee, ankle, shoulder, etc., an inflatable brace 102 suitably dimensioned and configured to support and immobilize that appendage may be applied to keep it from causing further injury due to movement. In other use cases, the inflatable brace may be configured to support other objects, such as fragile (non-human) objects during transportation/shipping, may be configured to support appendages of animals, or configured for use in other applicable applications. In some embodiments, the inflatable brace 102 may have an inflated state as shown in FIG. 1 and/or a deflated state such as the inflatable brace 102 shown in FIGS. 5A and 5B.

In the inflated state, the inflatable brace 102 may create a rigid structure using one or more inflatable channels that make up portions of the inflatable brace 102 that may be inflated and deflated. In some implementations, the inflatable brace 102 may not have any structural pieces that are permanently rigid (such as metal or plastic) but instead may include the one or more inflatable channels within the inflatable brace 102 that may be shaped such that when the inflatable channels are inflated they create rigidity around an object (such as the user 104) in the inflated state. In some implementations, the inflatable brace may include reinforcing members (e.g., rigid members made of metal, plastic, etc.) to complement the supportive aspects of the inflatable brace 102. For example, in a knee-brace, leg-brace, etc., application, medial and lateral rigid supports may be integrated with the inflatable brace to provide additional structural support, although further implementations may forgo such rigid structural members. In some implementations, hook and loop or other fastening straps may be included that wrap around and provide constricting pressure to the object being supported by the inflatable brace. Such straps may, in some cases, fasten to an outer surface of the brace material to position the straps. Other variations are also possible and contemplated.

The inflatable brace 102 may be easier to place around the appendage (e.g., neck) of the user 104 when compared to a rigid brace (e.g., rigid cervical collar). For example, in some implementations, when the inflatable brace 102 is in an uninflated state or low-inflation state, it may quickly and easily be manipulated and/or slid under a neck of a user 104 without jostling or moving the user 104. By not jostling or moving the user 104 when placing the inflatable brace 102 underneath the neck of the user 104, the risk of causing additional injury is avoided. The inflatable brace 102 may then be wrapped around the neck or other appendage of the user 104 in the uninflated state without causing any additional jostling of the user and/or applying any pressure to the neck or other areas of the user 104. The same is applicable to other objects-to-be-supported that are not easily moved during application.

The inflatable brace 102 may then be fastened in place. In some implementations, one side of the inflatable brace may loop around the appendage and fasten to the other side of the brace 102. In some implementations, the inflatable brace may comprises a fastener (including one or more fastening components) to couple one end of the inflatable brace 102 to the other. Example fasteners include but are not limited to buckles, straps, snaps, buttons, hook and loop, or any other suitable fasteners, some of which are described elsewhere herein. Any fastener capable of retaining the inflatable brace 102 around the object (e.g., retaining the collar around the neck of the user 104 before, during, and after inflation). The inflatable brace 102 may then be inflated as described elsewhere herein to a desired pressure until the inflatable brace 102 achieves rigidity in the one or more inflatable channels in the inflated state.

While in the inflated state, the inflatable brace 102 provides substantially even pressure around the neck as well as target areas, such as the shoulders and/or chin of the user to keep the neck and spinal area immobilized using the inflatable channels to achieve rigidity. In some configurations, such as with the collar depicted in FIG. 1, the inflatable brace 102 may advantageously apply even pressure around the immobilized area during inflation and when inflated. This helps to avoids causing further damage and/or pressure sores that can often result when using conventional braces (e.g., a rigid cervical collar for immobilizing a neck). In further implementations, the inflatable brace 102 when inflated allows for greater airflow when compared to a conventional brace because of the nature of the rigidity of the inflatable channels still allowing air flow through space created by the dividers.

In some examples, the inflatable brace 102 may be an example of one type of immobilization device that may immobilize the spine of a user by immobilizing movement of the user 104 at the neck. While in further embodiments, the immobilization device, may immobilize other areas of a user 104 or object (not shown) by inflating the inflatable collar around the area to immobilize the location and using pressure of inflatable channels of the inflatable brace 102 when in the inflated state to restrict movement around the desired area.

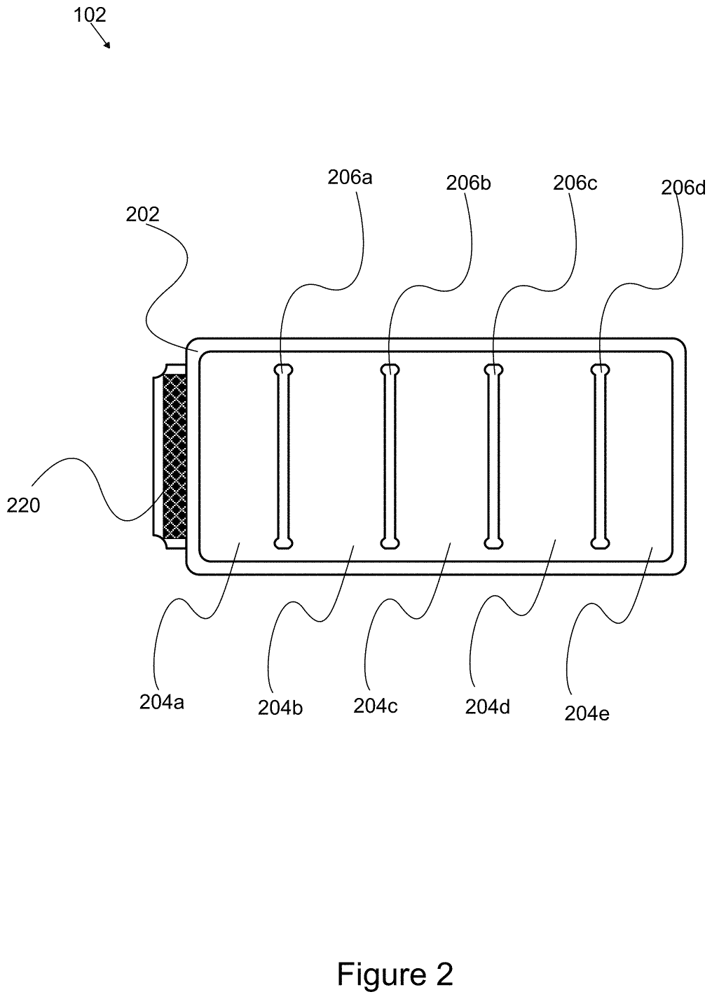

FIG. 2 shows an example embodiment of an inflatable brace 102. In some implementations, the inflatable brace 102 may be formed from a flexible and variably inflatable membrane 202. The membrane 202 may comprise one or more materials, such as a fabric or plastic that is flexible and may be rolled up for storage. The membrane may be multi layered. For example, the membrane 202 may comprise a first membrane layer situated on a second membrane layer that are sealed together to form an inflatable compartment or bladder that is airtight. Any suitable number of layers could be used, to form any suitable arrangement of inflatable compartments or channels. In this or other examples, a least a portion of a perimeter of the membrane may comprise a folded region of the membrane (e.g., one side may be folded onto another side of the membrane. Other variations are also possible and contemplated.

When the membrane 202 is unrolled or otherwise removed from a storage position, the membrane 202 may be unfolded and situated to lie substantially flat on a surface. In some implementations, the membrane 202 may be inflated using an inflation mechanism 514 (not shown), such as a pump. In some implementations, inflation may be variable, meaning that the arrangement of channels and sub-channels in the membrane may cause the channels and sub-channels to progressively inflate while the dividers that segment the channels and/or subchannels remain uninflated. Additionally, inflation may be variable meaning that the user may control the rate of inflation and/or the amount of fluid (e.g., air) with which to inflate the brace. For example, a user may select when to cause the membrane 202 to be inflated, such as by attaching an inflation mechanism and inflating the membrane 202. If the brace is equipped with an automated fluid pump, the pump may control the rate/amount of inflation medium. Further, a user and/or pump may also control the deflation of the membrane 202, such as by variably opening a valve to release an inflation medium from within the membrane 202. In some implementation, the user may also be able to control the amount of inflation medium being used to inflate the membrane 202 and may inflate the membrane 202 to different amounts of inflation as desired.

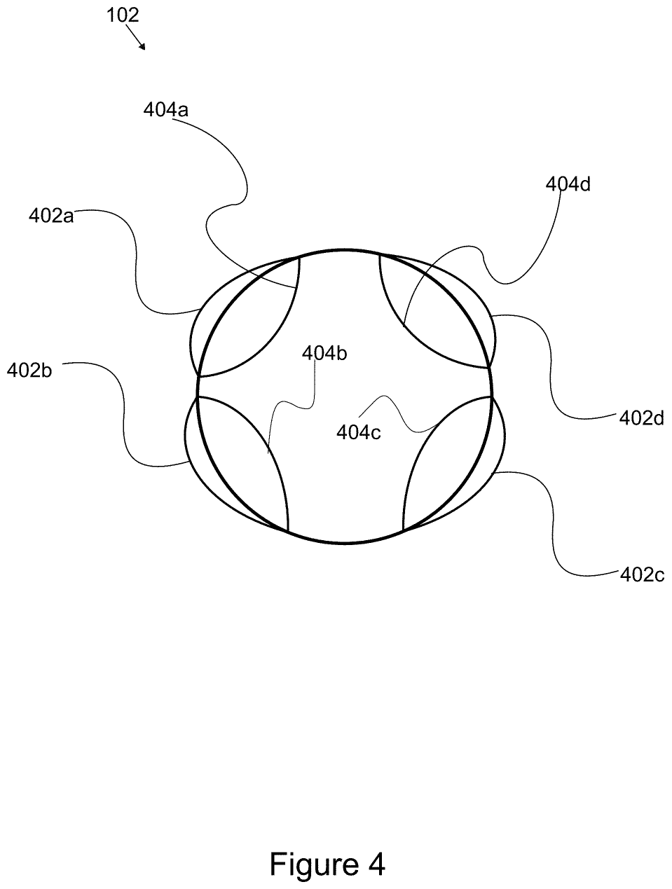

As shown in FIG. 2, the membrane 202 may include an inflatable channel 204 disposed between a first layer of the exterior surface 402 (not shown) of the membrane 202 and a second layer of the interior surface 404 (not shown) of the membrane 202, as shown with greater detail with respect to FIG. 4. The inflatable channel 204 may be airtight and retain an inflation medium, such as a gas or liquid when the inflation medium is placed in the inflatable channel 204. In some implementations, the membrane 202 may extend in a first direction longer than in a second direction. The membrane 202 may also extend in a third direction (e.g., z axis, thickness, etc.). For instance, the membrane may comprise an accordion-like edge region comprising a plurality of folds or joints that allow the membrane's thickness to expand when inflated, although other variations are also possible and contemplated.

In some implementations, the membrane 202 may include a plurality of inflatable channels and subchannels configured to provide support and/or rigidity when inflated. In some cases, the arrangement of segregated and inflatable sub-channels (such as inflatable channel 204a-204d in FIG. 2) may be predetermined. In some implementations, the sub-channels 204 may be segregated by a plurality of dividers 206. In some implementations, the predetermined arrangement of segregated and inflatable sub-channels 204 provide the particular structural support to an object when the membrane 202 is wrapped around the object based on the arrangement of the plurality of dividers 206 and/or the shape of the membrane 202.

In some implementations, the inflatable channel 204 may be formed by joining the layers of the membrane in particular regions. Any suitable manufacturing process for joining the layers of the membrane may be used, including application of adhesive, welding, bonding, application of pressure, etc. For example, the joints may be material welded according to a designated arrangement. More particularly, the joined regions may be joined to create the plurality of dividers 206 of a given arrangement that provides the structural properties of the brace when in the inflated state. In some examples, the inflatable brace 102 includes a thermoplastic polyurethane (“TPU”) material sandwiched between the first layer of the exterior surface 402 (not shown) of the membrane 202 and the second layer of the interior surface 404 (not shown) of the membrane 202. The welding may form welds making up the plurality of dividers 206 that joins together the TPU surfaces (first layer) and the outer surfaces (second layer) by heating the layers to the point of melting and pressing the layers together. Where the welds occur, there is no inflatable channel 204 to inflate and when the inflatable channel 204 does inflate, it will create an inflated structure.

As shown in FIG. 2, the inflatable brace 102 is in a deflated state. In the deflated state, the inflatable brace 102 may lack rigidity in one or more dimensions. For example, the inflatable brace may be unrolled and laid out substantially flat along a surface and/or be rolled up for storage in a compact fashion. In the deflated state, the inflatable brace 102 may be stored in a stuff sack or other appropriate storage location and take up much less space than a rigid cervical collar would. By taking up less space, a medical professional or other user using the inflatable brace 102 may be able to bring additional medical equipment and/or additional inflatable cervical collar(s) 102 to be more capable of treating patients in a medical situation. In some implementations, the inflatable brace 102 may be able to inflate and deflate easily for reuse.

In some implementations, the brace 102 may comprise a fastener 220. The fastener 220 may include one or more fastening devices, as described elsewhere herein. In some cases, the fastener 220 or a portion thereof may be integral (formed out of) or attached to the membrane 202, such as an end portion of the membrane 202. In some cases, a fastening device of the fastener 220 may be located on a left and/or right side of the membrane 202. As shown in the example in FIG. 2, a fastening device of the fastener 220 is located on the left side of the membrane 202. The fastener 220 may include a fastening/connection material (such as Velcro or another material that can couple two portions of the membrane 202 together). In some implementations, another connection tab of the fastener 220b may be located on an opposite side of the membrane 202 from the fastener 220 as shown with respect to FIG. 5A, while in further implementations, the connection tab may be located anywhere on an edge of the membrane 202. In some implementations, the fastener 220 may be configured to couple with the interior surface of the membrane 202 to further secure the inflatable brace 102 when in the inflated state and create a tubular shape that may surround the object to be immobilized.

For example, the fastener 220 may include compatible fastening devices (e.g., male/female fasteners, etc.), such as hook and/or loop (e.g., Velcro™) that may couple to the interior surface (which may include loop and/or hood) of the membrane 202 at any point along the interior surface. In some implementations, specific portions of the interior surface may include material designed to couple with the Velcro hook portions of the fastener. In some implementations, the fastener 220 may be strong enough such that only a portion of the fastener 220 needs to couple to the interior surface of the membrane 202 in order to stay connected in the inflated state and form the tubular shape. This allows for the inflatable brace 102 to be situated under a back of the neck of the user 104 and both sides of the membrane 202 may be wrapped around itself while wrapping around the neck of the user 104 such that the fastener 220 is on the inside loop of the wrap formed out of the membrane 202 and the fastener 220 makes contact with and couples to the interior surface as a result of being wrapped around itself

This may allow the inflatable brace 102 to have a variability in the sizes of the inner diameter and/or circumference of the inflatable brace 102 when the inflatable channel 204 is in the inflated state. In some implementations, if more of the inflatable brace 102 is wrapped around itself, then the inner diameter and/or circumference of the inflatable brace 102 will be smaller than if less of the inflatable brace 102 is wrapped around itself. Other fastening devices are also known and contemplated, such as using straps, hooks and loops, etc., to couple one side of the inflatable brace 102 to the other, and provide various/multiple sizing options. In some implementations, the inflatable brace 102 may additionally or alternatively include one or more slits, buckles, loops, or other devices that straps or other fastening devices may be threaded through to improve the fastening.

The variability of sizing allows the inflatable brace 102 to be used in a broad range of situations with a wide range of different sized users 104 compared to conventional braces, such as a rigid cervical collar that has to be sized to a specific patient. Furthermore, the nature of allowing the inflatable brace 102 to wrap around itself before being inflated allows for a quicker sizing of the patient than if a rigid cervical collar is used. By performing the sizing using the fastener 220 using a hook and loop (or similar material) allows for the inflatable brace 102 to be sized without putting pressure on the collar and/or user 104. Whereas, a rigid cervical collar may use straps to perform minor sizing adjustments or to create a stronger fit and the rigid cervical collar moves and jostles as pressure is applied to perform the minimal allowed sizing adjustments.

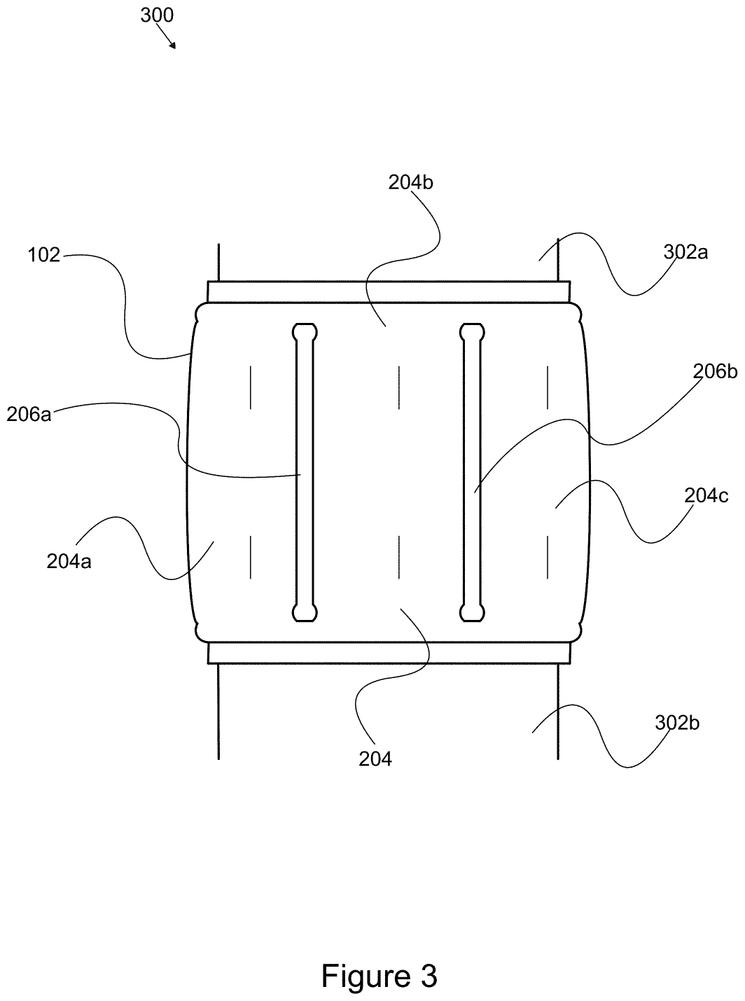

FIG. 3 is an example embodiment 300 of the inflatable brace 102 positioned around an object 302. In some implementations, the inflatable brace 102 may be wrapped around an object 302 in need of being immobilized and/or protected. The inflatable brace 102 may be wrapped around the object 302 when the inflatable channel 204 is in the deflated state. The inflatable brace 102 may then be wrapped around itself and fastened using the fastener 220 (not shown). The inflatable channel 204 may then be inflated around the object 302. As the inflatable channel 204 inflates, the dividers 206 may allow the inflatable channel 204 to inflate in a predetermined arrangement and the inflatable channel 204 may provide particular structural support to the object 302. In some implementations, the predetermined arrangement may allow for the inflatable channel 204 to provide particular structural support to a target area of the object 302. The target area may be an area where a specific type, direction, and/or amount of pressure in the form of the structural support may be applied to the target area of an object.

In one example, an object 302 may be a fragile item that needs to be protected, such as when the fragile item is going to be moved. The inflatable brace 102 may be stored in a rolled up and deflated state to minimize space until needed. The inflatable brace 102 may then be unrolled and positioned around the fragile item. In some implementations, the inflatable brace 102 may be positioned in a specific way around the fragile item to allow the inflatable channel 204 to provide the specific support to one or more target areas of the fragile item. The inflatable brace 102 may include instructions on a viewable exterior of the membrane 202 (not shown) that may guide a user on how to properly position the inflatable brace 102 around the fragile item. The inflation channel 204 may then be inflated to a specific pressure and the inflation channel 204 may provide the particular structural support to the fragile item that allows the fragile item to be protected and not be damaged.

FIG. 4 shows a top view of an example embodiment of the inflatable brace 102 fastened around itself. In some implementations, the inflatable brace 102 may include a first layer forming an exterior surface 402 of the membrane 202 and a second layer forming an interior surface 404 of the membrane 202. The inflation channel 204 may be disposed between the first layer and the second layer such that when the inflation channel 204 is filled with an inflation medium (e.g., air, helium, CO2, etc.), the exterior surface 402 and the interior surface 404 expand as a result of the pressure caused by the inflation medium.

In some implementations, the exterior surface 402 may have an elasticity that causes the exterior surface 402 to stretch while still remaining air-tight based on an elasticity of the exterior surface 402. In some implementations, the interior surface 404 may have an elasticity that causes the interior surface to stretch while still remaining air-tight based on an elasticity of the interior surface 404. In some implementations, one surface may have a significantly different elasticity than the other (e.g., greater than 5%, greater than 10%, etc.). For example, the interior surface 404 may have a greater elasticity than the exterior surface 402 that may cause the interior surface 404 to stretch further when compared to the exterior surface as shown in the top-down view of FIG. 4. In some implementations, the interior surface 404 may also have different materials and or weaves that cause the interior surface 404 to stretch more in a horizontal direction than in a vertical direction. By having a greater elasticity on the interior surface 404 of the membrane 202, the inflatable brace 102 expands inward around an object without deforming the tubular shape created by the exterior surface 402 which allows the particular structural support to be applied evenly to target areas.

In some implementations, having the exterior surface 402 be less elastic allows the interior surface 404 to stretch more during inflation and expand inward. By expanding inward, the inflatable brace 102 may maintain the shape created by the exterior surface 402 while providing even support around the object being immobilized as the interior surface 404 expands. Furthermore, by stretching inward, the exterior surface 402 provides for more contouring and creating of the structure, such as the shelf 510 (not shown) as described elsewhere herein.

In some examples, the exterior surface 402 may comprise a 70 denier airtight ripstop nylon. The ripstop nylon may be a woven fabric that is resistant to tearing and/or ripping. During weaving, thicker reinforcement threads are interwoven at intervals in a crosshatch pattern. In some examples, the interior surface 404 may include a 2-way stretch fabric that allows the material to stretch both horizontally and vertically. In further examples, the interior surface 404 may stretch different vertically than it does horizontally, such as stretching more horizontally than the material stretches vertically. The difference in the stretching may allow for a difference in rigidity in the two directions and by stretching less vertically, the inflatable brace 102 may be more rigid in the vertical direction. In some examples, the interior surface 404 may comprise a 2-way polyurethane stretch material. The stretching of the interior surface 404 may allow the inflatable channels 204 created by the dividers 206 to stretch evenly to fill the space around the object (such as a neck of a user 104).

FIG. 5A shows an example embodiment of an inflatable brace 102 from a front view. In some implementations, the front view may show the exterior surface 402 of the membrane 202. In some implementations, the inflatable brace 102 may include contouring rather than straight lines around the edges to allow the inflatable brace 102 to be positioned around various objects. For example, the inflatable brace 102 may be configured to wrap around a neck of a patient and the edges of the membrane 202 may curve up where the patient's shoulders are located to allow the inflatable brace 102 to fit around the neck and provide structural support to the neck by bracing against the chest, back, chin, and/or head.

As shown in the example in FIG. 5A, the dividers 206 may be positioned in the predetermined arrangement to create the segregated inflatable sub-channels. In the depicted predetermined arrangement the dividers may be specifically spaced and have varying lengths between them to create the segregated inflatable sub-channels. For example, dividers 206a-206h may be positioned on a left side of the membrane 202 while divider 206i may be positioned on a right side of the membrane 202, allowing for when the membrane 202 is wrapped around the object and fastened using the fastener 220, the dividers 206 may be distributed around the membrane 202 forming the tubular shape in the predetermined arrangement and creating rigid structure when inflated.

In some implementations, some of the dividers 206 may be connected into further arrangements beyond straight lines, such as u-shaped dividers 508. As shown in FIG. 5A, the membrane 202 may include u-shaped dividers 508 that are formed to resemble an upside-down “u-shape”. In some examples the u-shaped dividers 508a and 508b may be located on the left and/or right side of an opening forming the access 512. In some implementations, the u-shaped dividers 508 may be shaped like upside down horseshoes with the rounded portions on the upper portion connecting the two vertical components of the horseshoe shape. In some implementations, the u-shaped dividers 508 may be specifically designed to prevent the inflatable brace 102 from folding and providing additional structural support when in the inflated state. In further implementations, other shapes of dividers are contemplated and used based on the contribution of the shape to providing structural support to the object when inflated.

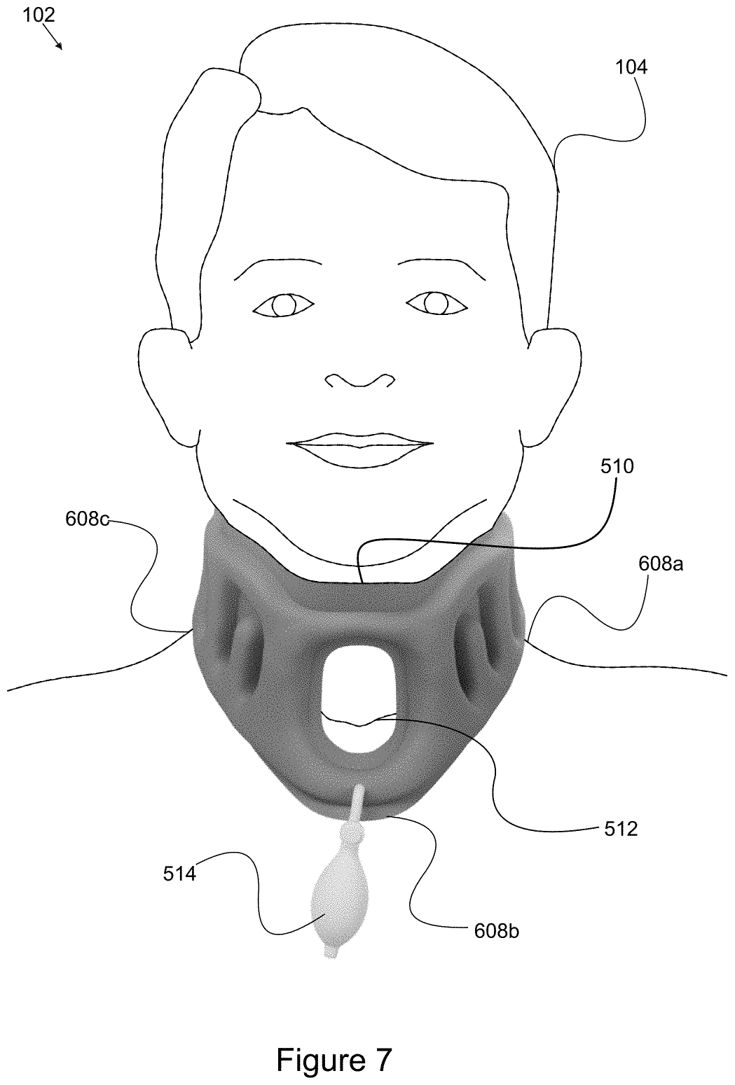

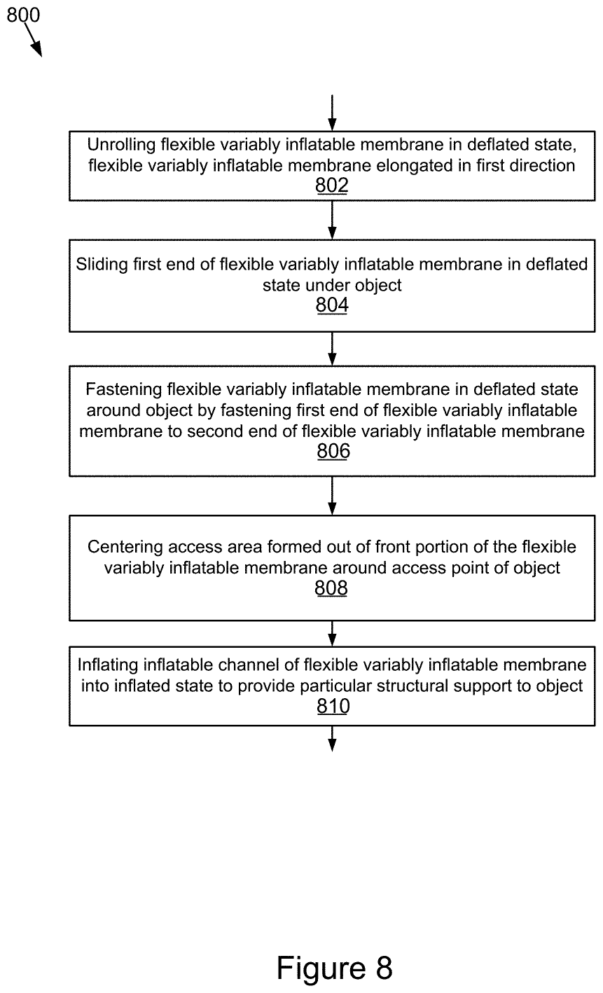

As shown in FIG. 5A, the u-shaped dividers 508 may be located below a shelf 510 on the front of the inflatable brace 102. The shelf 510 may be a portion formed from the first layer and/or the second layer of the membrane 202 that may be configured to support a protrusion of an object when the inflatable appendage brace is wrapped around an object. For example, the inflatable brace 102 may be wrapped around a tree that has been damaged and cannot remain standing without support and the shelf 510 may be configured to support a branch of the tree protruding from the trunk. In another example, when the inflatable brace 102 is wrapped around a user's neck, the users chin may protrude out beyond a portion of the membrane 202 and the shelf 510 may be adapted to support and retain the chin of the user. In some implementations, an extra lip of non-inflated fabric may be located along a top edge of the shelf 510. The extra lip may wrap around the front of the chin of the user to help position and stabilize the neck of the user 104 as shown in FIGS. 1 and 7. In some implementations, the shelf 510 may be configured to extend outward to accommodate where the jaw of the user 104 extends out from the neck as shown from a side view in FIG. 4 and provide support along the jaw line and around the chin of the user 104.

The inflatable brace 102 may include the access 512 for accessing a portion of the object when the inflatable brace 102 is wrapped around the object. In one example, the access 512 may be located on the inflatable brace 102 such that when the inflatable brace 102 is in the inflated state on a user 104, the access 512 is located at the center and directly below the chin of the user 104 around the tracheal area. The access 512 may allow for access to the throat of the user 104 during emergent situations. In further implementations, the access 512 may be used to interact with the object while the inflatable brace 102 is wrapped around the object. For example, the inflatable brace 102 may be wrapped around a fragile electronic device and the access 512 may allow a user to interact with an input and/or display of the fragile electronic device while the fragile electronic device is protected by the inflatable brace 102.

In some implementations, the inflatable brace 102 may include one or more valves 516 for connecting an inflation mechanism 514 to inflate in the inflatable channel 204 of the inflatable brace 102. In some implementations, the valve 516 may be a two-way valve where the valve 516 allows a desired inflation medium (such as air, liquid, gas, etc.) to be inflated or inserted into the inflatable channels 204 to create a rigid structure. In further implementations, the valve 516 may allow the inflatable brace 102 to be deflated when desired.

In some implementations, the valve 516 may be located on a center of the inflatable brace 102 in the inflated state. By locating the valve 516 on the center area, as the inflatable brace 102 is inflated to the inflated state, the valve 516 allows for even distribution of the inflation medium (e.g., air, gas, liquid, etc.) as the inflatable brace 102 inflates around the object.

In some implementations, the valve 516 may be located at an edge, such as the bottom (bottom center of the inflatable brace 102 as shown in the figures) of the inflatable brace 102 which may improve the deflation of the inflatable brace 102 by forcing the inflation substance to the edge with the valve 516. In some implementations, if the valve 516 is located at a bottom edge, then the valve 516 may advantageously be prevented from protruding and/or jabbing into parts of the user when in the inflated state. In some implementations, the inflatable brace 102 and/or the inflation mechanism 514 may include a pressure sensor and the inflation mechanism 514 may automatically stop inflating the inflatable channels 204 of the inflatable brace 102 when a detected pressure of inflation is reached.

In some implementations, the inflation mechanism 514 may comprise a pneumatic pump (such as hand pump, electric and/or battery powered pump, gas powered pump, etc.), or pressurized fluid release device (e.g., CO2 canister and release mechanism), or any other suitable device, that may be coupled to the valve 516 to inflate and/or deflate the inflatable brace 102 into the inflated state and/or deflated state. The inflation mechanism 514 may be configured to be attached and/or be removed from the valve 516. In some examples, a medical responder may carry multiple inflatable appendage braces 102 in the deflated state (to conserve space) and a single inflation mechanism 514 to inflate and/or deflate one or more of the inflatable appendage braces 102. In some implementations, the inflatable brace 102 may be configured to be inflated without using a pneumatic pump, such as by inflating it by breathing air through the valve, etc.

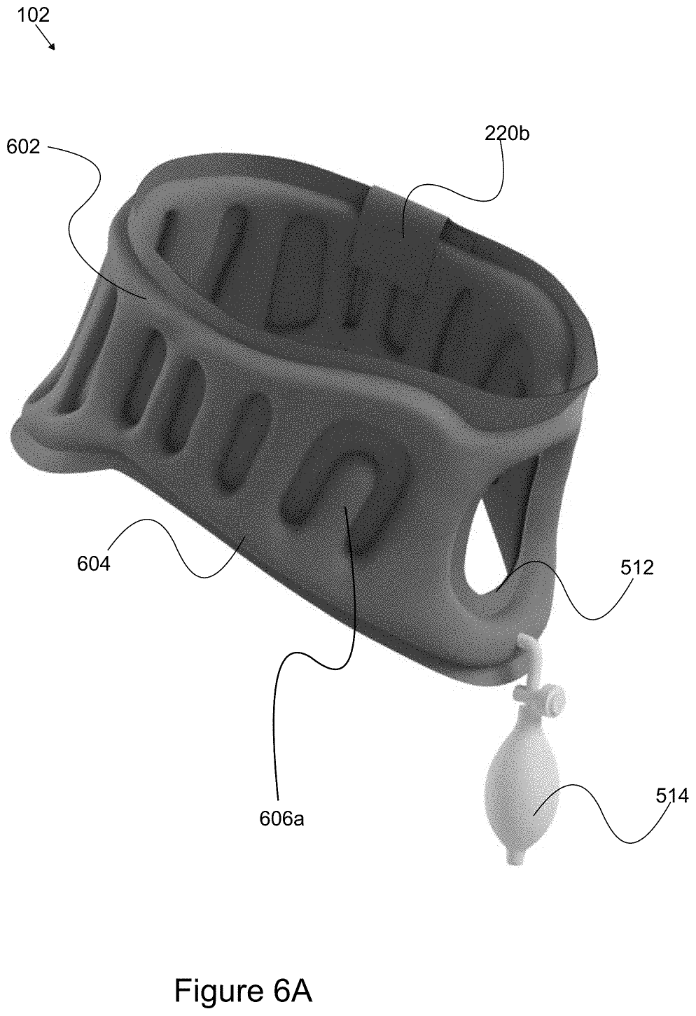

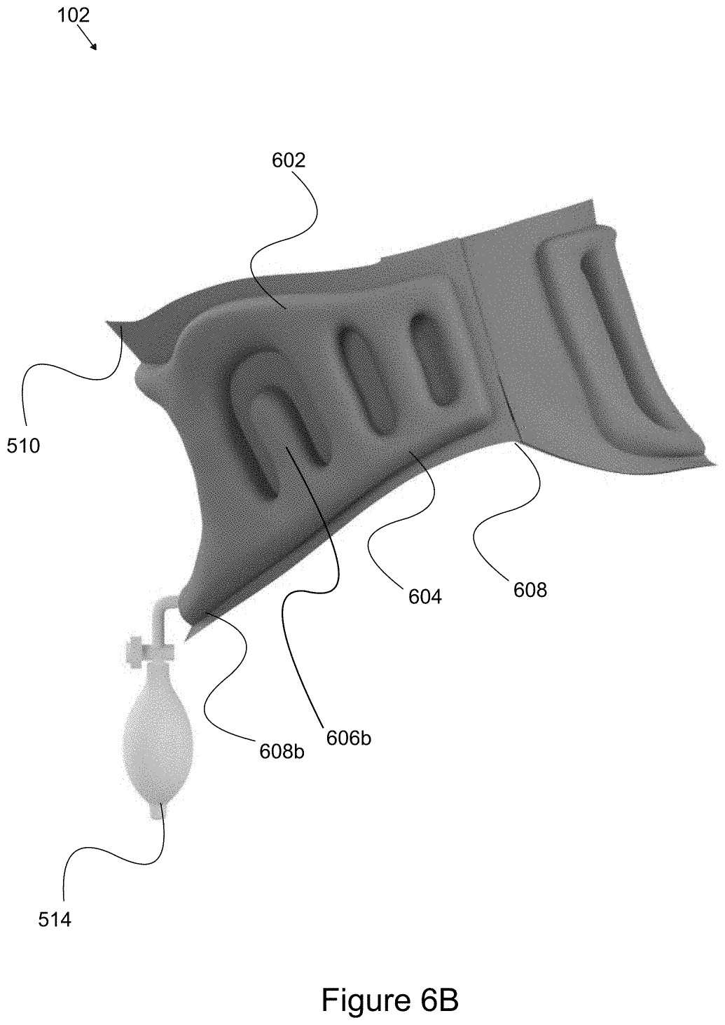

In some implementations, the membrane 202 may include a second fastener 220b that may comprise one or more fastening devices. In some cases, the second fastener 220b may form a connection tab. In some implementations, the connection tab may be configured to fold over the top and/or bottom edge of the inflatable brace 102 and connect to the interior surface 404 and/or the fastener 220 on the interior portion of the membrane 202. By folding over the fastener 220, the connection tab may provide an additional secure coupling to the interior surface 404 and/or the fastener 220 and may apply a coupling force at a different direction when compared to the fastener 220 as shown in FIG. 6A.

FIG. 5B shows an example embodiment of a back view of the inflatable brace 102. The back view may show the interior surface 404 of the membrane. As described elsewhere herein, in some implementations, the interior surface 404 may be formed of a different material with a different elasticity. As shown in FIG. 5B, the dividers 206 and/or the u-shaped dividers 508 may be visible from both the front view and the back view and may be welded together from the first layer and the second layer. In some implementations, the valve 516 may include reinforcement on the interior surface such that the valve does not puncture the membrane 202.

FIG. 6A shows an example embodiment of the inflatable brace 102. In the example, the inflatable brace 102 is inflated and the inflation channels 204 are shown forming the particular structural support. In the example, the inflation channels 204 include vertical sub-channels that run vertically in a second direction between the vertical dividers 206. In some implementations, the inflation channel 204 may also include a top horizontal sub-channel 602 that runs along a top of the inflatable brace 102 in the first direction and above the dividers 206. In some implementations, the inflation channel 204 may also include a bottom horizontal sub-channel 604 that runs along a bottom of the inflatable brace 102 in the first direction and below the dividers 206. The top horizontal sub-channel 602 and the bottom horizontal sub-channel 604 may provide particular structural support to the object in a direction different than the vertical sub-channels 204 to create a more particular structural support. In some implementations, the inflation channel 204 may include a horseshoe shaped sub-channel 606 that inflates within the u-shaped divider. In some implementations, all of the sub-channels may be configured to inflate evenly from a single vale 516 (e.g., a single access point and not separate valves).

In the example, the connection portion of the fastener 220b of the inflatable brace 102 is shown from a side view. As depicted in the drawing, the fastener 220b is coupled to the other side of the inflatable brace 102 when wrapped in the inflated state. The fastener 220 may be able to connect closer to or farther from the rear side of the inflatable brace 102 as described elsewhere herein to change the dimensions of the inflatable brace 102 for sizing.

FIG. 6B shows inflatable brace 102 from a side-view. In some implementations, the top horizontal sub-channel may be configured to support a head of a user when the inflatable brace 102 is wrapped around the neck of a user. The top horizontal sub-channel 602 may inflate to create specific contouring to support the head of the user. In some implementations, target areas of the user may be identified when forming the predetermined arrangement of the sub-channels. For example, a portion of the user's jaw may be identified as needing increased structural support to prevent movement and the top horizontal sub-channel 602 may be larger and/or contoured to surround a portion of the target area (e.g., jaw in this example) and provide increased structural support.

In some implementations, the bottom horizontal sub-channel 604 may inflate to create specific contouring to support target areas. For example, the target area 608a and 608b may be portions where the bottom horizontal sub-channel 604 may be contoured and adapted to provide increased structural support. For example, target area 608a may be adapted to conform around the shoulder area of a user and target area 608b may be a support area on the chest of the users that may provide structural support to the inflatable brace 102 to provide the structural support. The bottom horizontal sub-channel 604 may create a uniform inflated area around the top and bottom edge of the inflatable brace 102 when in the inflated state that may apply even pressure to the neck and shoulders of the user and include target areas such as the chest and or back where inflatable pressure points may be adapted to apply pressure and support the structure.

FIG. 7 shows an example embodiment of an inflatable brace 102 positioned on a user 104 from a front perspective. As shown in the example, the access 512 may be an opening that allows access to a neck of the user 104 through the access 512. As shown and described elsewhere herein, the shelf 510 may provide support for a protrusion of an object, such as a chin of the user 104 in this example. In some implementations, the shelf 510 may also be configured to apply pressure in a target area to provide support for the inflatable brace 102. In some implementations, the shelf 510 may include loose, uninflated material that may provide additional support to the protrusion without inflating to jostle and/or move the protrusion. As shown in the example and described elsewhere herein, the target areas 608 may be areas where the particular structural support is applied to retain the object. In some implementations, the inflatable brace 102 may be designed to accommodate and create structure around shoulders of the user 104 when in the inflated state, by causing the rear portion to extend below the shoulders to the back of the user 104 and a front portion that extends down onto a chest of the user 104 and creates pressure points at target areas (such as the chest and/or jaw).

In some implementations, instructions (not shown) for correctly placing the inflatable brace 102 around the neck of the user 104 may be printed on the exterior of the membrane 202. For example, an arrow and/or instructions indicating to position the arrow underneath the right ear of the user 104 may be printed on the exterior. As shown, the various welds may be specifically designed to create varying dimensions of inflatable channels for creating rigidity in the inflated state. In some implementations, the length of the welds and the margins of the endpoints are important dimensions between the welds, as depicted in the Figures.

FIG. 7 shows a flowchart 800 of an example method of applying and/or inflating an inflatable brace 102. At 802, a user may unroll a flexible variably inflatable membrane 202 in a deflated state. In some implementations, the flexible variably inflatable membrane 202 may be elongated in a first direction such that the flexible variably inflatable membrane 202 is long enough to fasten around an object.

At 804, a user may slide a first end of the flexible variably inflatable membrane 202 in the deflated state under an object. In some implementations, the flexible variably inflatable membrane 202 may be substantially flat and may be positioned under an object without jostling and/or moving the object. For example, the object may be a person with a potential neck injury, and the first end of the flexible variably inflatable membrane 202 may be slid under the neck of the person without moving the person's head and potentially causing further injury.

At 806, the user may fasten the flexible variably inflatable membrane 202 in the deflated state around the object by fastening the first end of the flexible variably inflatable membrane 202 to a second end of the flexible variably inflatable membrane 202 which forms a tubular shape around the object. As described elsewhere herein, the fastening may be done using a fastener such as Velcro or other fasteners as described elsewhere herein. At 808, the user may center an access area formed out of a front portion of the flexible variably inflatable membrane 202 around an access point of the object. For example, the access area may be a hole in the flexible variably inflatable membrane 202 and the user may position the access area in front of the neck of the patient.

At 810, the user may use an inflation mechanism 514 to inflate an inflatable channel that is air-tight and formed out of a first layer of the flexible variably inflatable membrane 202 and a second layer of the flexible variably inflatable membrane 202. The user may inflate the inflatable channel into an inflated state at a desired pressure level and the flexible variably inflatable membrane 202 may provide particular structural support to the object.

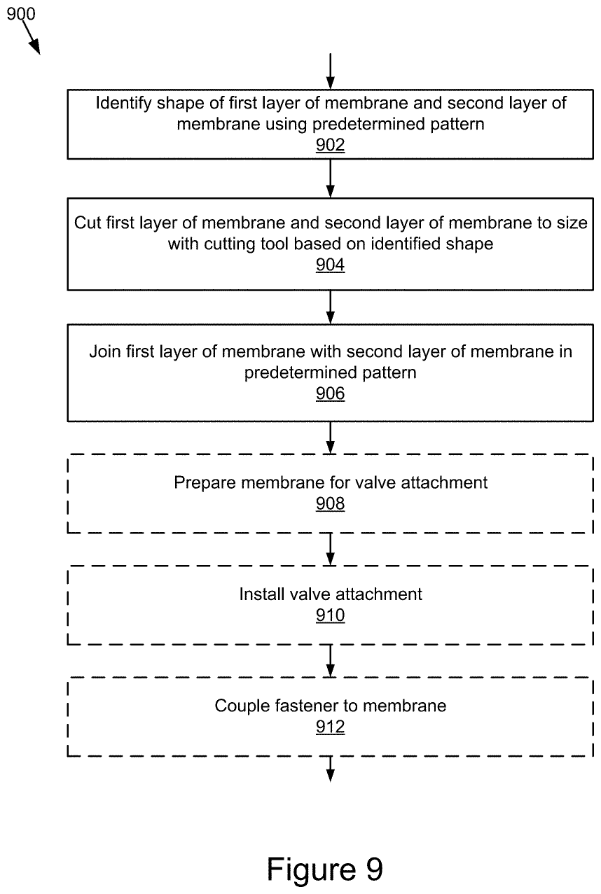

FIG. 9 is a flowchart 900 of an example method of manufacturing an inflatable brace 102. At 902, a design for the layers of the membrane 202 (e.g., a shape of a first layer of the flexible variably inflatable membrane 202 and a shape of a second layer of the flexible variably inflatable membrane 202) may be identified by a manufacturing computing system and/or a user. In some implementations, the shape of the first layer and the shape of the second layer may be the same shape, while in further implementations, the shape of the first layer and the shape of the second layer may be different. In some implementations, the first layer and the second layer may be connected and folded over each other to create two layers. In one example, the first layer may include additional contours in the outline that allow for the flexible variably inflatable membrane 202 to provide particular structural support to an object when assembled. In some implementations, the shape of the first layer and the second layer may be identified by accessing manufacturing instructions that provide specific measurements of a shape for the first layer and/or the second layer. In further implementations, various inflatable braces may be manufactured with different structural properties based on the different shapes of the first layer and/or the second layer and the instructions provide an indication of which shape to use to cut the first layer and/or the second layer.

At 904, a cutting tool may cut the first layer of the flexible variably inflatable membrane 202 and/or the second layer of the flexible variably inflatable membrane 202 to the size indicated based on the identified shape. In some implementations, the cutting tool may be automated and a machine may be configured to receive the identified shape and use a blade (such as a knife and/or scissor blade) to cut the material in the identified shape. In further implementations, the cutting tool may be a manual tool that a user may manipulate to cut the material of the first layer and/or the second layer, such as scissors or a sharp blade that may cut the material.

At 906, the first layer of the flexible variably inflatable membrane 202 and the second layer of the flexible variably inflatable membrane 202 may be joined. In some implementations, the first layer and the second layer are joined based on a predetermined pattern. In some implementations, a material welding tool may weld the first layer and the second layer may be joined using material welding. The material welding may involve heating specific portions of the first layer and the second layer with a heating element, laser, frequencies, and/or other techniques, and bonding the heated layers together at the specific portions such that they attach and remain connected forming an airtight seal. In some implementations, the first layer and the second layer may be laminated and then exposed to a press using heat, ultrasonic, radio frequency, or another hot roller using various welding methods and the first layer and second layer may be welded together into the predetermined pattern forming the various dividers as described elsewhere herein. In some implementations, the outer edges of the first layer and second layer may be sealed by material welding the two layers together to create an airtight seal between the first layer and the second layer. In some implementations, excess material on the outside of the welded bond may be removed, such as with a cutting tool.

At 908, the flexible variably inflatable membrane 202 may be prepared for a valve attachment. In some implementations, the valve 516 as described elsewhere herein may be a two-way valve that as affixed to at least one layer of the inflatable membrane and the valve attachment may be the entire valve. In further implementations, the valve attachment may be a component that a valve 516 may be connected to and form a seal such that an inflation medium within the flexible variably inflatable membrane 202 cannot exit through the valve attachment unless the valve 516 is manipulated to allow the inflation medium to exit. At 910, the valve attachment may be installed. In some implementations, the valve attachment may be installed directly into the flexible variably inflatable membrane 202. In some implementations, the valve attachment may be separate from the valve 516, while in other implementations valve 516 and the valve attachment may be a single component. In one example, one of the layers of the flexible variably inflatable membrane 202 may be cut open at a specific location and valve attachment may be installed. In some implementations, the area around the valve attachment may be sealed, such as with further material welding to insure that the flexible variably inflatable membrane 202 remains airtight.

In some implementations, as shown at 912, the fastener 220 may be coupled to the flexible variably inflatable membrane 202. For example, the fastener 220 may include Velcro or another type of fastener and the Velcro may be attached to a surface of the flexible variably inflatable membrane 202, such as by sewing and/or material welding the Velcro. In some implementations, the fastener may be webbing strips that may be attached, such as by sewing, and slits may be formed in the flexible variably inflatable membrane 202 that the webbing strips may be attached to in order to fasten the flexible variably inflatable membrane 202 to itself. In further implementations, the slits in the flexible variably inflatable membrane 202 may be reinforced, such as through sewing and/or material welding.

Various aspects related to an inflatable brace have been described. In the above description, for purposes of explanation, numerous specific details are set forth in order to provide a thorough understanding of the techniques introduced above. It will be apparent, however, to one skilled in the art that the techniques can be practiced without these specific details. In other instances, structures and devices are shown in block diagram form in order to avoid obscuring the description and for ease of understanding. For example, the techniques are described in one embodiment above primarily with reference to specific examples. However, the present invention applies to any type of inflatable appendage brace.

It should be understood that any suitable automated manufacturing tools, CNC tools, robots, conveyors, spools, material cutting devices, computing devices with non-transitory memories, processors, and networking components, etc., may be used to manufacture braces or portions thereof. In further case, some aspects may be manually performed.

Reference in the specification to “one embodiment” or “an embodiment” means that a particular feature, structure, or characteristic described in connection with the embodiment is included in at least one embodiment. The appearances of the phrase “in one embodiment” in various places in the specification are not necessarily all referring to the same embodiment.

The foregoing description of the embodiments has been presented for the purposes of illustration and description. It is not intended to be exhaustive or to limit the specification to the precise form disclosed. Many modifications and variations are possible in light of the above teaching. It is intended that the scope of the embodiments be limited not by this detailed description, but rather by the claims of this application. As will be understood by those familiar with the art, the examples may be embodied in other specific forms without departing from the spirit or essential characteristics thereof. Likewise, the particular naming and division of the modules, routines, features, attributes, methodologies and other aspects are not mandatory or significant, and the mechanisms that implement the description or its features may have different names, divisions and/or formats. Additionally, the specification is in no way limited to embodiment in any specific material, or for any specific situation or environment. Accordingly, the disclosure is intended to be illustrative, but not limiting, of the scope of the specification, which is set forth in the following claims.

What is claimed is:

1. An inflatable brace comprising:
a flexible variably inflatable membrane elongated in a first direction and a second direction, the flexible variably inflatable membrane being fastenable around an object and inflatable to provide particular structural support to the object; and
an inflatable channel disposed between a first layer of the variably inflatable membrane and a second layer of the variably inflatable membrane, the inflatable channel comprising a predetermined arrangement of segregated inflatable sub-channels that are segregated by a plurality of dividers, the arrangement of segregated inflatable sub-channels, when inflated, providing the particular structural support to the object.

2. The inflatable brace of claim 1, further comprising;
an inflation mechanism coupled to the flexible variably inflatable membrane, the inflation mechanism causing the inflatable channel to be inflated.

3. The inflatable brace of claim 2, wherein the inflation mechanism is located on a front side of the variably inflatable membrane and configured to provide symmetrical inflation of the inflatable channel.

4. The inflatable brace of claim 1, wherein the plurality of dividers includes at least one u-shaped divider that prevents folding of the flexible variable inflatable membrane when the inflatable channel is inflated.

5. The inflatable brace of claim 1, wherein the predetermined arrangement of segregated inflatable sub-channels includes at least one of a first horizontal inflatable sub-channel and a second horizontal inflatable sub-channel,
the first horizontal inflatable sub-channel being disposed along a top portion of the flexible inflatable membrane in the first direction and adjacent to a top of at least a portion of the plurality of dividers, and
the second horizontal inflatable sub-channel being disposed along a bottom portion of the flexible inflatable membrane in the first direction and adjacent to a bottom of at least a portion of the plurality of dividers.

6. The inflatable brace of claim 1, wherein the flexible variably inflatable membrane further comprises:
an opening such that a target area of the object can be accessed through the opening when the flexible variably inflatable membrane is fastened around the object.

7. The inflatable brace of claim 1, wherein the flexible variably inflatable membrane further comprises:
a shelf formed out of a portion of a top edge of the flexible variably inflatable membrane, the shelf configured to provide specific structural support to retain a protrusion of the object.

8. The inflatable brace of claim 1, wherein the inflatable channel is configured to provide particular structural support to a target area of the object.

9. The inflatable brace of claim 1, wherein the flexible variably inflatable membrane is fastenable around the object using a fastener, the fastener being coupled to a first end portion of the variably inflatable membrane and configured to couple with a second end portion of the variably inflatable membrane and retain the variably inflatable membrane in a tubular shape around the object.

10. The inflatable brace of claim 9, wherein the fastener is one of a Velcro patch and webbing configured to be threaded through a slit of the variable inflatable membrane.

11. The inflatable brace of claim 1, wherein the first layer of the variably inflatable membrane comprises a first material having a first elasticity and the second layer of the variably inflatable membrane comprises a second material having a second elasticity greater than the first elasticity, the second elasticity providing for an increased stretching of the second material when the inflatable channel disposed between the first layer and the second layer is inflated.

12. The inflatable brace of claim 11, wherein the second material having the second elasticity allows for the second material to stretch more in the first direction of the variably inflatable membrane than in the second direction.

13. The inflatable brace of claim 1, wherein the flexible variably inflatable membrane is configured to lie substantially flat when the inflatable channel is uninflated.

14. The inflatable brace of claim 1, wherein the flexible variably inflatable membrane further comprises:
instructions displayed on an exterior portion of the flexible variably inflatable membrane, the instructions including information on how to use the inflatable brace.

15. The inflatable brace of claim 1, wherein the flexible variably inflatable membrane is capable of being rolled up on itself for storage when the inflatable channel is uninflated.

16. A method of using an inflatable brace, the method comprising:
unrolling a flexible variably inflatable membrane in a deflated state, the flexible variably inflatable membrane being elongated in a first direction;
sliding a first end of the flexible variably inflatable membrane in the deflated state under an object;
fastening the flexible variably inflatable membrane in the deflated state around the object by fastening the first end of the flexible variably inflatable membrane to a second end of the flexible variably inflatable membrane;
centering an access area formed out of a front portion of the flexible variably inflatable membrane around an access point of the object; and
inflating an inflatable channel of the flexible variably inflatable membrane into an inflated state to provide particular structural support to the object, the particular structural support being formed by a predetermined arrangement of segregated inflatable sub-channels that are segregated by a plurality of dividers.

17. An inflatable brace comprising:
a flexible variably inflatable membrane elongated in a first direction and a second direction, the flexible variably inflatable membrane being fastenable around a neck of a user and inflatable to provide particular structural support to the neck of the user; and
an inflatable channel disposed between a first layer of the variably inflatable membrane and a second layer of the variably inflatable membrane, the inflatable channel comprising a predetermined arrangement of segregated inflatable sub-channels that are segregated by a plurality of dividers, the plurality of dividers including a plurality of vertical dividers and one or more u-shaped dividers, the arrangement of segregated inflatable sub-channels, when inflated, providing the particular structural support to the neck of the user.

18. The inflatable brace of claim 17, wherein the flexible variably inflatable membrane further comprises:
an opening that provides access to the neck of the user when the flexible variably inflatable membrane is inflated.

19. The inflatable brace of claim 17, wherein the flexible variably inflatable membrane is fastenable around the neck of the user using a fastener, the fastener being coupled to a first end portion of the variably inflatable membrane and configured to couple with a second end portion of the variably inflatable membrane and retain the variably inflatable membrane in a tubular shape around the neck of the user.

20. The inflatable brace of claim 17, wherein the first layer of the variably inflatable membrane comprises a first material having a first elasticity and the second layer of the variably inflatable membrane comprises a second material having a second elasticity greater than the first elasticity, the second elasticity providing for an increased stretching of the second material when the inflatable channel disposed between the first layer and the second layer is inflated.

21. A method of manufacturing an inflatable brace, the method comprising:
identifying a first shape of a first layer of a flexible variably inflatable membrane and identifying a second shape of a second layer of a flexible variably inflatable membrane using a predetermined pattern;
cutting, with a cutting tool, the first layer and the second layer to the shape identified by the first shape and the second shape;
joining the first layer with the second layer in the predetermined pattern;
preparing the flexible variably inflatable membrane for a valve attachment; and
installing the valve attachment.
